(19) United States
(12) Patent Application Publication
(10) Pub. No.: US 2019/0385741 A1
(43) Pub. Date: Dec. 19, 2019

(54) SYSTEMS AND METHODS FOR PROVIDING HEALTH CARE USING AN INTEGRATED DELIVERY PLATFORM

(71) Applicant: HOY HEALTH LLC, Morristown, NJ (US)

(72) Inventors: Mario R. ANGLADA CORTES, Morristown, NJ (US); José Miguel FEBUS RODRIGUEZ, San Juan, PR (US)

(21) Appl. No.: 16/439,486

(22) Filed: Jun. 12, 2019

Related U.S. Application Data
(60) Provisional application No. 62/684,447, filed on Jun. 13, 2018.

(51) Int. Cl. G16H 50/20; G06N 5/04; G06Q 30/02

(52) U.S. Cl. CPC G16H 50/20; G06Q 30/0237; G06N 5/04

(57) ABSTRACT

The disclosed technology relates to a platform for providing improved access to medication, increasing medication adherence, and managing medical conditions. The platform includes one or more servers connected via a network. The servers are configured to access one or more databases that are configured to store data associated with a user, voucher data, and one or more medications. The platform also includes an analytics module for analyzing the data stored in the databases. The analytics module generates a medical treatment recommendation based on the user and/or voucher data. The platform also includes a voucher module for generating vouchers that may be redeemed for prescribed medication and tracks redemption of the vouchers.

### CROSS-REFERENCE TO RELATED APPLICATION

The present application claims the priority benefit of U.S. patent application No. 62/684,447, filed Jun. 13, 2018, entitled “SYSTEMS AND METHODS FOR PROVIDING HEALTH CARE USING AN INTEGRATED DELIVERY PLATFORM,” the disclosure of which is incorporated herein by reference.

### TECHNICAL FIELD

The present disclosure relates generally to systems and methods for providing healthcare using an integrated delivery platform, and more particularly to a healthcare system and method that provides improved access to medication, improves medication adherence, and manages medical conditions via a platform.

### BACKGROUND

Treatment of a medical condition may involve separate parties, such as medical doctors or specialists, clinics, hospitals, and/or pharmacies, that may lead to the fractured treatment of a patient's medical condition. As a result, a patient may have trouble complying with treatment plans and/or adhering to medication prescriptions, which may ultimately lead to a worsening of the medical condition for which treatment was originally sought.

In addition, patients may experience extreme medication price variability that may be unknown to a patient until a prescription is attempted to be filled. As a result, non-adherence to a recommended treatment plan may further worsen a medical condition. Moreover, some methods for obtaining prescribed medication require payment for the medication at a retail outlet, such as a pharmacy location, at the time a prescription is filled thereby making it difficult for others, such as family members, to pay for the prescribed medication.

### SUMMARY

According to various aspects of the subject technology, a healthcare system and method that provides improved access to medication, improves medication adherence, and manages medical conditions via an integrated delivery platform is provided. According to aspects of the subject technology, a computer-implemented method for generating a recommendation to treat a medical condition is disclosed. The method includes analyzing data associated with a user to generate a first recommendation for medically treating a medical condition; obtaining treatment data from a client device representing a characteristic of the user; and generating a second recommendation for medically treating the medical condition based on the treatment data.

Another aspect of the present disclosure relates to a non-transient computer-readable storage medium having instructions embodied thereon, the instructions being executable by one or more processors to perform a method for generating a recommendation to treat a medical condition. The method may include analyzing data associated with a user to generate a first recommendation for medically treating a medical condition; obtaining treatment data from a client device representing a characteristic of the user; and generating a second recommendation for medically treating the medical condition based on the treatment data.

Yet another aspect of the present disclosure relates to a system configured for generating a recommendation to treat a medical condition. The system may include one or more hardware processors configured by machine-readable instructions. The processor(s) may be configured to analyze data associated with a user to generate a first recommendation for medically treating a medical condition; obtain treatment data from a client device representing a characteristic of the user; and generate a second recommendation for medically treating the medical condition based on the treatment data.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The embodiments herein may be better understood by referring to the following description in conjunction with the accompanying drawings in which like reference numerals indicate identical or functionally similar elements. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates a conceptual block diagram illustrating an example network environment utilizing a healthcare platform, in accordance with various aspects of the subject technology;

FIG. 2A illustrates an example graphical user interface of a healthcare platform, in accordance with various aspects of the subject technology;

FIG. 2B illustrates an example voucher generated by a healthcare platform, in accordance with various aspects of the subject technology;

FIG. 3 illustrates a kiosk of a healthcare platform, in accordance with various aspects of the subject technology;

FIG. 4 illustrates an example process for generating a recommendation to treat a medical condition, in accordance with various aspects of the subject technology;

FIG. 5 illustrates an example of a system configured to treat a medical condition using an integrated delivery platform, in accordance with some aspects.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

The disclosed subject matter describes systems and methods for providing healthcare using an integrated delivery platform. Data associated with users may be analyzed to generate a digital record of each user. The digital record may be used to effectively engage respective users and address their unique medical needs. A recommendation based on the user data and/or digital record may be generated to identify medication, testing supplies, and/or devices that may treat a medical condition of the user. Additional data may be collected from a client device associated with the user during treatment to refine or revise the treatment recommendations. In another aspect, data associated with a plurality of medications may be presented to the user for purchase. If purchased, a voucher is provided to the user for redemption at participating pharmacies. In yet another aspect, a kiosk may provide the user with the voucher. The kiosk may also provide the user with access to a medical advisor via a display, camera, speaker, and microphone, at the time of medication purchase and voucher acquisition.

The disclosed technology addresses the need in the art for more effective patient care technologies by utilizing a platform to collect and aggregate data from disparate sources to generate a recommendation to treat a medical condition, and to utilize new data collected from a client device associated with the user to improve adherence to the recommendation. The platform is configured to transmit the recommendation to the client device associated with the user and in response, the client device is configured to execute instructions associated with the recommendation to remind, prompt, or encourage the user to follow the recommendation (e.g., take medication, engage in physical activity, take a test, etc.), gather data regarding a health characteristic of the user (e.g., movement data, blood test data, user weight data, etc.), and transmit data to the platform for further analyzing and generation of a second recommendation; thereby improving medication adherence and management of medical conditions.

These and other embodiments address various technical problems in the computing field as well. For example, the various approaches taken address the shortcomings in the various application program interfaces (APIs) and/or proprietary data structures provided by disparate consumer products that collect user data, such as wearables, pedometers, blood pressure reading devices, blood test sample devices, scales, heartrate monitors, and other devices that may be configured to collect and present certain data to the user, but do not make such data available to other devices or platforms. Structural limitations of such data further prevent robust querying of data sets. Additionally, the various approaches further reduce the bandwidth and processing time required to perform various functions.

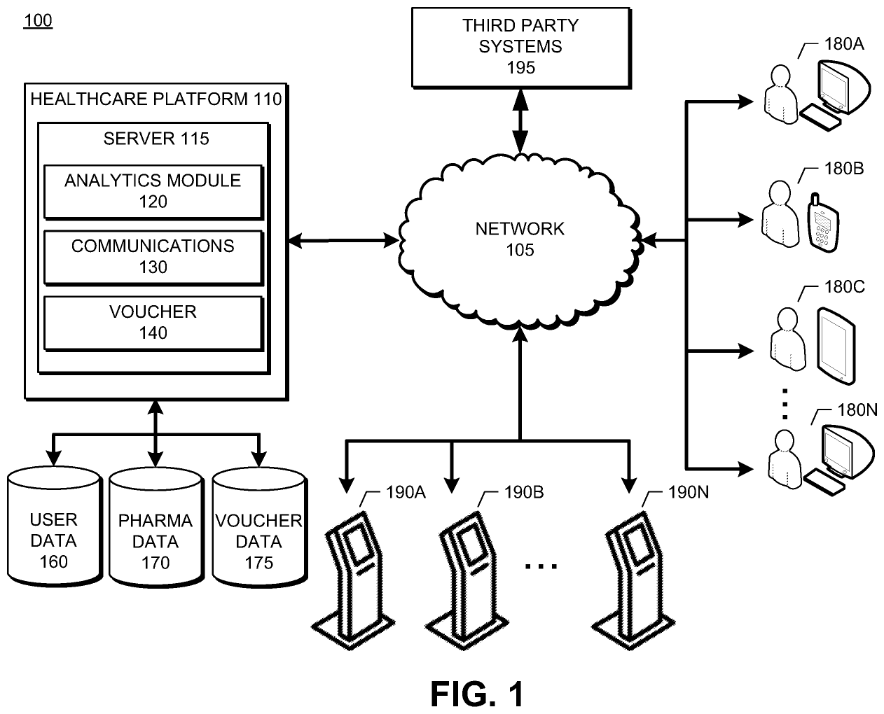

FIG. 1 illustrates a conceptual block diagram illustrating an example network environment 100 utilizing a healthcare platform 110, in accordance with various aspects of the subject technology. The integrated delivery platform 110 may comprise a one or more servers 115 connected via a network 105. The one or more servers 115 may be configured to communicate with one or more client devices 180A-N according to a client/server architecture and/or other architectures. Users may access the platform 110 via the client devices 180A-N. The servers 115 may be configured by machine-readable instructions. Machine-readable instructions may include one or more instruction modules. The instruction modules may include computer program modules. The instruction modules may include one or more of an analytics module 120, a communications module 130, a voucher module 140, a billing module 150, and/or other instruction modules.

The one or more servers 115 may be any system or device having a processor, a memory, and communications capability for providing content to the client device 180A-N. In some example aspects, the one or more servers 115 can be a single computing device such as a single computer server. In other embodiments, the one or more servers 115 can represent more than one computing device working together to perform the actions of a server computer (e.g., cloud computing).

The network 105 can include, for example, any one or more of a cellular network, a satellite network, a personal area network (PAN), a local area network (LAN), a wide area network (WAN), a broadband network (BBN), the Internet, and the like. Further, the network 105 can include, but is not limited to, any one or more of the following network topologies, including a bus network, a star network, a ring network, a mesh network, a star-bus network, tree or hierarchical network, and the like.

The one or more servers 115 may be configured to access a database 160 that is configured to store data associated with a user. User data may include, for example, data relating to prior medication purchases, medical records from healthcare organizations, medical condition, biometric data, genetic data, health literacy data, telehealth consultation data, wearable device data, medication adherence data, family composition data, email, contact information, client device information (e.g., MAC address), address, chat data, supplement and vitamin use, retail purchase data, condition predictive algorithm data, health condition status, and/or chat bot data. Each user may be keyed to a user ID and the user database 160 may include information for client devices 180A-N associated to a particular user.

In some aspects, user data may be stored in accordance with regulations, such as HIPAA. As such, data stored in user database 160 may be segmented such that data that may identify a user may be removed from medical data. In such an example, minimal user data (e.g., (user ID, first and last name, email, date of birth, and/or address) may be stored in a HIPAA compliant data store, and data relating to prior medication purchases, medical records from healthcare organizations, medical condition, biometric data, genetic data, health literacy data, telehealth consultation data, wearable device data, medication adherence data, family composition data, supplement and vitamin use, retail purchase data, condition predictive algorithm data, health condition status, and/or chat bot data may be stored in an anonymous data store. The data in the anonymous data store may be keyed by the user ID which represents a unique alpha-numeric or numeric code (e.g., 339fj3-33d4-fkfkf-33e3) that yields no identifying information on its own.

The one or more servers 115 may also be configured to access a pharmaceutical database 170 that is configured to store data associated with a plurality of medications, including information relating to, for example, chemistry, dosage, price (including usual and customary pricing, discount pricing, and savings), trade names, generic names, and medication type.

Data in the user database 160 may be populated in batch processes by querying one or more third-party data providers and/or gradually by storing information provided by the client device 180A-N over time. According to some embodiments, data is available to the platform 110 and may be replicated and stored locally in order to address the technical problem caused by disparate systems that do not provide sophisticated APIs and backend functionality that enable fast and efficient querying. In other aspects, storing user data in database 160 allows for more complex queries, which allows for more efficient and powerful segmentation and targeting of the users that will receive a treatment recommendation. For example, should a new medication be discovered that has better results over prior medications, targeting users who may benefit from the new medication based on a particular medical condition can be performed more quickly and efficiently.

The analytics module 120 may be configured to analyze data stored in the one or more databases 160, 170. In one aspect, the analytics module 120 may generate a digital record of the user that may be used to effectively engage users and address their unique medical needs. Data stored in the database 160, 170 and/or analyzed by the analytics module 120 may be stored and transmitted in a secure block chain. In other aspects, the analytics module 120 may generate a medical treatment recommendation based on the user data and/or digital record. The treatment recommendation may, for example, include a recommendation for medication, testing supplies, and/or devices that are intended to treat a chronic medical condition of the user. Devices that may be recommended by the analytics module may include peripherals, such as heart monitors, blood pressure monitors, scales, or other devices that may be used to collect information or data about a user and transmit the collected information or data to the platform 110 for storage in the database 160 and analyzing by the analytics module 120.

The communications module 130 may be configured to communicate with a user via the client device 180A-N. The client device 180A-N may be capable of running an application and communicating with the platform 110, to provide the user with access to a healthcare provider for further treatment of the chronic medical condition. The client device 180A-N may be a mobile phone, PDA, portable media player, tablet, laptop, or other appropriate computing device, and may utilize a touch sensitive user interface, such as a touch-sensitive screen, to receive user input. The touch screen of the client device 180A-N may be built into the device itself, or can be electronically connected to the device (e.g., as a peripheral device). The user input may comprise gestures or touch. In some example aspects, the client device 180A-N may be any machine with appropriate hardware/software to launch and run one or more applications or software.

One or more of the applications may include application data comprising a graphical user interface. The application may thus, be configured to receive user input using the graphical user interface and the touch-sensitive screen. The application's graphical user interface may include touch elements that are displayed to the user and configured to trigger an application function based on user input.

In one aspect, the platform 110 transmits the medical treatment recommendation to the client device 180A-N to encourage the user to follow the recommendation (e.g., take medication, engage in physical activity, take a test, etc.); gather data regarding the user, their health, and compliance with the recommendation; and transmits the data back to the platform 110 for further analyzing thereby improving medication adherence and management of medical conditions.

The voucher module 140 may be configured to communicate with the client device 180A-N and provide to the client device 180A-N information associated with the plurality of medications. The information associated with the plurality of medications is displayed at the client device 180A-N to provide to the user information about the one or more medications of the plurality of medications. Such information displayed to the user may relate to a price of the one or more medications.

The platform 110 may be configured to display to a user a usual and customary price of one or more medications with a discounted price for the one or more medications, as well as a difference between the two, to inform a user of savings associated with the one or more medications. In one aspect, the disclosed technology addresses the need in the art for providing more information to consumers to enable consumers to make informed purchasing decisions regarding medications. Conventional systems do not provide consumers with savings information and/or customary and usual pricing information, making it difficult for consumers to appreciate potential savings. Disclosure of such information in a graphical user interface at the time of purchase increases medication adherence rates as consumers are able to budget for prescribed medications more effectively.

Data representing the customary and usual price for medications may be gathered by the platform 110 by querying one or more third party databases (e.g., pharmacy benefits manager database). The data representing the customary and usual price may be stored in the pharmaceutical database 170 for querying and generation of a graphical user interface. Data representing a discounted price for medications may be gathered by the platform 110 from one or more pharmacy databases. The data representing the discounted price may be stored in the pharmaceutical database 170 for querying and generation of the graphical user interface.

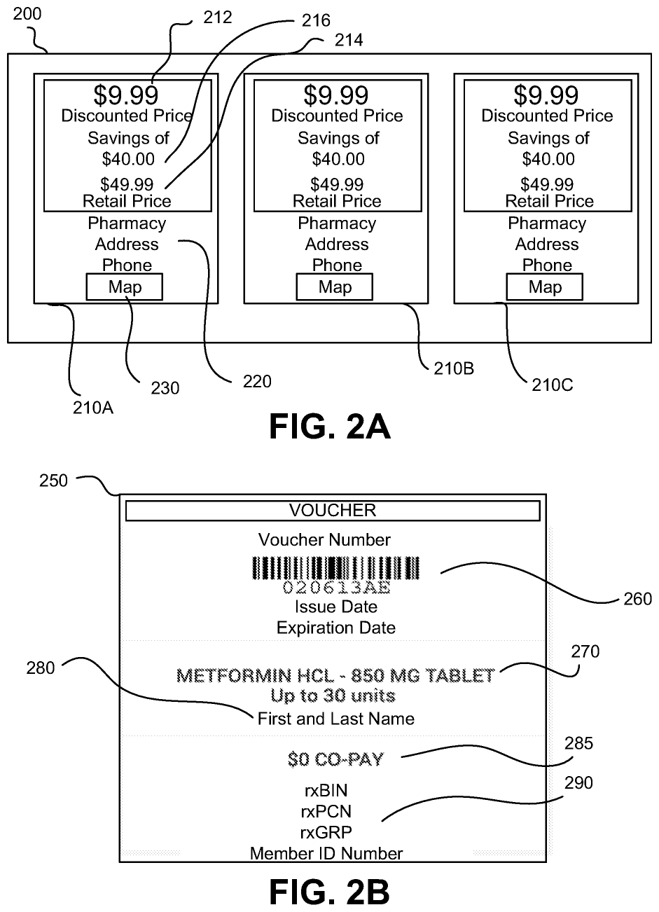

FIG. 2A illustrates an example graphical user interface 200 of a healthcare platform, in accordance with various aspects of the subject technology. The graphical user interface 200 pricing information for one or more medications based on data stored in the pharmaceutical database 170. The graphical user interface 200 comprises a plurality of boxes 210A-C that each display pricing information for a medication based on different pharmacies. Each box 210A-C includes a discount price 212, a customary and usual price 214, and a savings amount 216 which may be displayed as a percentage of savings over the usual and customary price 214 or a difference in amount between the usual and customary price 214 and the discount price 212. Each box 210A-C also displays a name, address, and phone number or other contact information 220 for each pharmacy. Each box 210A-C may also include one or more buttons that are configured to perform an operation, such as a mapping operation 230, when pressed or touched. In one aspect, the pricing information displayed in each box 210A-C may utilize different colors to further denote and differentiate the discount price, savings, or usual and customary price from each other. For example, the savings 216 may be denoted in a green color, the usual and customary price 216 may be denoted in a red color, and the discount price 212 may be denoted in a blue color. Other colors are of course contemplated without departing from the scope of the subject technology. In some aspects, by providing the pricing information (e.g., discounted price 212, usual and customer price 214 and/or savings 216) for multiple pharmacies in a single graphical user interface 200, a consumer or user can discern pricing information easily over conventional systems.

Referring back to FIG. 1, should a user wish to purchase the one or more medications, the platform 110 receives payment confirmation that the user has completed a transaction. In response, the voucher module 140 generates and provides to the client device 180A-N a voucher to enable the user to obtain the one or more medications. In some aspects, the voucher is configured to be redeemed at participating pharmacies without requiring further payment from the user.

FIG. 2B illustrates an example voucher 250 generated by a healthcare platform, in accordance with various aspects of the subject technology. The voucher 250 may include a unique code or ID 260 to prevent fraud, expiration date (e.g., 90-day validity), issue date, purchased medication information 270 (e.g., medication name, dosage, number of units, refills, etc.), user information 280 (e.g., purchaser name or intended recipient name), copay amount 285 (if any), and information that may be used by a pharmacy to verify medication information 290 (e.g., medication bin number, product code, group insurance number, insurance member number, etc.). In some aspects, by providing pricing information to a user for medications, a user receives certainty relating to prescription costs thereby improving medication adherence. In other aspects, by allowing any user to access the platform 110 via a client device 180A-N, any user may purchase medication for themselves, or others, thereby improving medication adherence and access to medication.

Referring back to FIG. 1, in some aspects, to obtain prescribed medications, a user simply needs to execute three steps: (1) search; (2) pay; and (3) redeem voucher. For example, a user may search for available medications by accessing a portal through a web browser application running on the client device 180A-N. The user may, for example, search via brand name, generic, conditions, drug categories, or top most sold drugs. The data displayed to the user may also be provided in different languages, such as English and Spanish. In some example aspects, the voucher module 140 and or one or more servers 115 of the platform 110 may provide application data and content for display on the client device 180A-N. The content can include a graphical user interface, such as the graphical user interface 200 of FIG. 2A. The content can also include text or a web link. Of course, other types of content can also be provided. In some example aspects, the content can be transmitted from the one or more servers 115 or voucher module 140 via the network 105 to the client device 180A-N. In other example aspects, the content can be stored in a storage component (e.g., hard disk, RAM, ROM, etc.) of the respective client device 180A-N.

Once a desired medication is found, the user may purchase the medication for themselves or others, as desired by engaging in a “checkout” process. Upon purchase, the user immediately receives a voucher from the voucher module 140 having a unique voucher ID that grants the user the ability to redeem the voucher at a pharmacy. The voucher may be printed or displayed for redemption via the client device 180A-N. In some aspects, the voucher is unique to the user, the purchase, and/or the medication purchased. Data associated with the voucher may be stored in a voucher database 175. The voucher data may comprise user ID to identify a user, customer ID to identify a person to which the voucher is for, unique voucher ID to identify the voucher, purchase ID to identify the financial transaction purchasing the medication, medication ID to identify the medication, dosage information identifying the dosage and/or quantity, refill information indicating the number of refills, and redemption code to track voucher redemption. In one aspect, voucher data stored in voucher database 175 may be stored so that no user identifying information is stored with the voucher data, thereby enabling the voucher data to remain anonymous. A user ID may be used to key the voucher data, with the user ID comprising a unique alpha-numeric code or numeric code that yields no identifying information on its own.

In one aspect, the voucher ID may comprise a hash number comprising a block chain that includes user information, medication information, and purchase information. The hash number provides anonymity to the user, in compliance of regulations that may require anonymity, yet enables the tracking of a voucher, confirmation of payment for a medication, and identification of the medication purchased. The hash data further includes information identifying the prescribed dosage for the medication. In one aspect, the hash number represents a stored value enabling conversion of the hash value into a monetary amount. As such, the platform 110 is configured to structure data representing user data and voucher data into a unique hash number that is referred to herein as the voucher ID. In this example, once the voucher is redeemed, the block chain is updated to indicate that the voucher has been redeemed.

To redeem the voucher, a pharmacy may enter or scan the unique voucher ID assigned to the voucher that may be provided in a printout or as an image on the client device 180A-N. The voucher ID may be submitted to the platform 110 by the pharmacy by, for example, accessing the platform 110 via a web browser, terminal, point of sale, or third party system 195 connected to the platform 110 via the network 105. The platform 110 validates the voucher ID to ensure that the voucher is valid and may, for example, utilize the voucher database 175 to validate the voucher ID. If validated, the platform 110 transmits a confirmation to the pharmacy or retail outlet and stores a code in the redemption code data field in the voucher database 175 to denote that a particular voucher has been redeemed. The platform 110 further deems the voucher ID as “closed” for no further use.

In one aspect, a user may purchase a voucher for another person by identifying the intended recipient during purchase. For example, the user may identify the person during “checkout” by name, ID, address, email, phone number, or other identifying information. In response, the platform 110 stores the information and assigns the customer ID to the intended recipient. The purchased voucher may also be reassigned to another user for gifting by the purchasing user, as desired. In addition, vouchers may be cancelled for a refund or reissued for different medication in the event of an error by the purchasing user.

In one aspect of the subject technology, the platform 110 is configured to track each issued voucher for redemption by using the redemption code. Data associated with redemption (e.g., redemption data) may be used by the analytics module 120 to generate a treatment recommendation. For example, the redemption data may be used by the platform 110 to incentivize user redemption and/or increase medication adherence. For example, the platform 110 may perform a query for unredeemed vouchers and send a message to the corresponding users' client devices 180A-N to remind them that a voucher has not yet been redeemed. In another example, the platform 110 may query the voucher database 175 to identify redeemed vouchers that are approaching a refill and in response, transmit a message to the client devices 180A-N to remind users of upcoming refills. In another example, the platform 110 may identify an unredeemed voucher by running a query against data stored in the voucher database 175, and generate and transmit to the corresponding client device 180A-N a new treatment recommendation that is based on non-redemption of a voucher, and consequently, a user's non-adherence to prescribed medication. The new recommendation may, for example, comprise alteration of the medication associated with the non-redeemed voucher to increase or decrease dosage, change medication, or other alteration to improve treatment of the user's medical condition.

User database 160 and voucher database 175 may be shared with third party systems 195, such as a user's healthcare professional or provider to further improve medication adherence and treatment. Data stored in the user database 160 and/or the voucher database 175 may be filtered based on user ID. The platform 110 may be configured to receive a query from third party systems 195 based on user ID and in response, queries the user database 160 and/or the voucher database 175. The platform 110 may be configured to provide the third party system 195 with access to data associated with the user ID. For example, a user's healthcare provider may submit a query to the platform 110 to confirm whether the user has redeemed a voucher for prescribed medicine. The platform 110 may query the voucher database 175 and may provide to the third party system 195 redemption data associated with the user or voucher. In another example, the platform 110 may be configured to query the voucher database 175 and in response, calculate a redemption metric (e.g., a percentage of redeemed vouchers vs. non redeemed vouchers) for a particular user or group of users (e.g., based on age, gender, location, ethnicity, medical condition, etc.), and may further filter such results based on the medication or medication type (e.g., diabetes medicines, pain management, heart medication, etc.). In some aspects, data stored in the voucher database 175 may be queried based on medication or medication type (e.g., diabetes medicines, particular diabetes medicine, pain management, heart medication, etc.) to enable third party systems 195 to gain insights as to redemption percentages or rates for a group of users.

In other aspects, the platform 110 may provide statistics to the user to incentivize adherence to a treatment plan. For example, the platform 110 may query the user database 160 and/or the voucher database 175 to calculate a percentage of redemption rates of other users in a similar or same peer group (e.g., same or similar location, medical condition, age, gender, ethnicity, etc.) to inform the user how others are performing in comparison to the user. By generating and storing voucher-related data, data representing voucher redemption may be used to improve healthcare, incentivize redemption, and improve medical care for the user.

In some embodiments, the platform 110 may provide an interface (e.g., an API) that enables the client device 180A-N and data-gathering peripherals, such as heart monitors, blood pressure monitors, scales, or other devices that may be used to collect information or data about a user, to receive a request for data from the platform 110, to transmit data to the platform 110, and/or retrieve a recommendation generated by the analytics module 120.

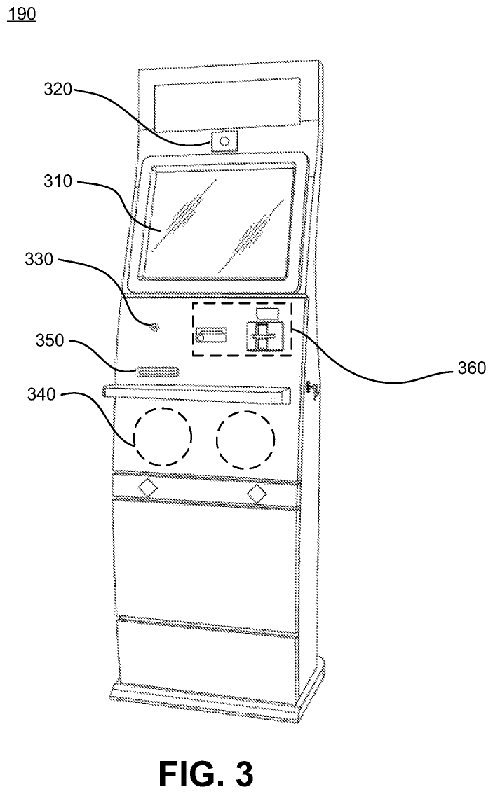

FIG. 3 illustrates a kiosk 190 of a healthcare platform, in accordance with various aspects of the subject technology. The network environment 100 may include a number of kiosks 190A-N communicably connected to one or more servers 115 by the network 105. Each of the kiosks 190A-N may include a touch screen 310 for receiving user input and displaying information associated with the one or more medications of the plurality of medications, a camera 320 for capturing an image or video, a microphone 330 for receiving audio input, a speaker 340 for transmitting audio, a printer 350 for providing a paper voucher to the user, and a bill acceptor or card reader 360 for accepting payment. In another aspect, each kiosk 190A-N may be configured to provide a user with access to a medical advisor via the display, camera, speaker, and microphone, at the time of medication purchase and voucher acquisition. The kiosks 190A-N may include a processing device and a data store. The processing device executes computer instructions stored in the data store, for example, to present information regarding the one or more medications, receive payment, print the voucher, connect with a medical advisor, capture video, transmit video, capture audio, transmit audio, display video and play audio.

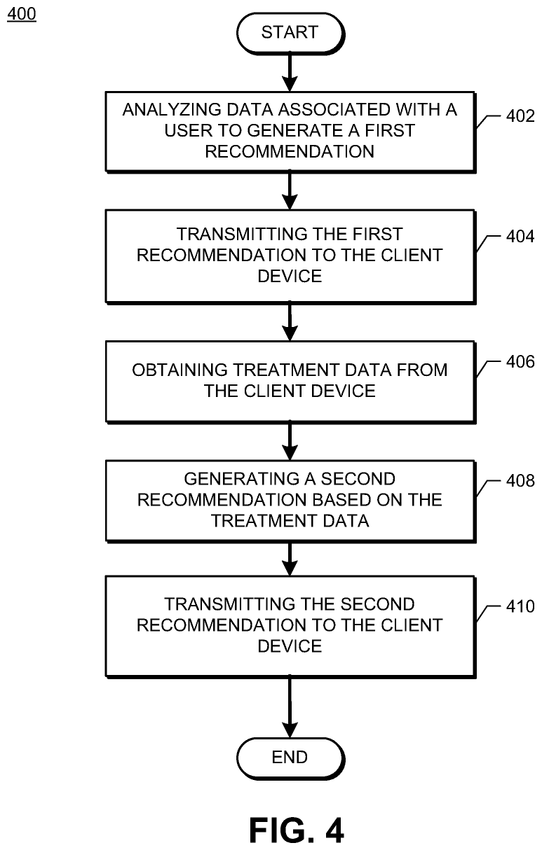

FIG. 4 illustrates an example process 400 for generating a recommendation to treat a medical condition, in accordance with various aspects of the subject technology. Following start block 400, a user accesses the platform via a client device. The user may create an account and grant the platform access to the user's medical records, health data, wearable data, family history data, and/or medication data. The data may be stored in a database accessible by the platform for analyzing and generation of a recommendation for medically treating a medical condition.

At step 402, an analytics module of the platform accesses and analyzes the data associated with the user and generates a first recommendation for treating a medical condition. The first recommendation may include at least one of a medical prescription (e.g., generating a voucher as described above), testing device (e.g., blood test kits, blood pressure monitoring, insulin testing kits, etc.), telemedicine (e.g., remote patient monitoring), peer mentoring (e.g., same age, gender, race, ethnicity, religion, language, medical condition as user), and data-gathering peripherals (e.g., mobile devices, apps, fitness trackers, etc.).

For example, for a user suffering from high cholesterol, the analytics module may access a user's prior cholesterol levels, weight, and age to generate a recommendation for treating high cholesterol that may include a prescription for a drug to lower cholesterol, a blood testing kit to monitor cholesterol levels, telemedicine for remote patient monitoring, assignment of a peer mentor (having the same age, gender, and ethnicity as the user, as well as having or having had high cholesterol), and a pedometer to use in tracking movement and activity of the user. The platform may identify a number of steps the user should take every day to help lower the user's cholesterol and may utilize data provided by the pedometer to track the user's daily progress. The pedometer may be configured to provide movement data to the platform for further analyzing and generation of a new recommendation. The blood testing kit may also be configured to test a user's blood and obtain a cholesterol level that is subsequently transmitted to the platform for further analyzing and generation of a new recommendation.

At step 404, the first recommendation generated by the platform may be transmitted to the user's client device. The recommendation may instruct the client device to transmit data back to the platform for monitoring the user's adherence to the recommendation. In one example, the recommendation may be utilized by an application operating on the client device to provide notifications to the user to remind or prompt the user to engage in a physical activity, take medication at the appropriate time, take tests, or provide feedback. The recommendation may comprise a set of instructions that are read by the client device to perform operations, such as displaying reminders, prompts, or feedback, as well as instructions to direct the client device to transmit data (e.g., movement data, test results, etc.) to the platform.

At step 406, data collected by the client device (e.g., treatment data) is transmitted to the platform. In one aspect, data gathered or collected by the client device may also include data provided by peripherals (e.g., blood pressure testing device, pedometer, activity tracking device, wearables, scale, etc.) that are configured to communicate with the client device via a wired or wireless connection. Data collected by the client device may comprise movement data, testing data, sleeping pattern data, weight data, and/or other data associated with a user and indicative of a characteristic of the user. Data transmitted to the platform from the client device may be stored in the user database 160 for analysis and generation of a refined or new recommendation to treat the medical condition. For example, the platform may be configured to monitor redemption data to confirm whether the user has redeemed a medical voucher for access to a prescribed drug. If the platform determines that the voucher has not been redeemed, the platform may generate a recommendation that causes the client device to remind the user to redeem the voucher to gain access to medicine to treat the medical condition. In another example, should the voucher be redeemed, the platform may generate a recommendation that informs the user via the client device that a new voucher should be obtained to refill their medicine.

At step 408, the platform generates a second recommendation for treating the medical condition based on data received from the client device. The platform may, for example, receive treatment data comprising weight data and movement data and may compare the treatment data to previously received treatment data associated with the user to determine that the user has lost weight and is active and in response, may generate a third recommendation for a reduced dosage of medication. In this example, the platform may issue a voucher for the reduced dosage to enable the user to obtain the recommended medicine and dosage. The platform may monitor subsequent blood test results to confirm a desired downward trend for the cholesterol levels of the user and continue to reduce dosage to prevent over-dosing of the user.

At step 410, subsequent recommendations generated by the platform may be transmitted to the user's client device to aid in monitoring the user's progress in treating their medical condition. As such, the platform is configured to continually or periodically reassess the health of the user based on received treatment data to develop a customized and dynamic treatment plan to achieve a desired result and a healthier user.

In another example, a user may be diabetic and in need of medical care to reduce blood sugar. The platform may access the user's prior blood glucose, weight, medication history, ethnicity, family history and age to generate a recommendation for treating high blood sugar that may include a prescription for a drug to lower blood sugar, a blood testing kit to monitor glucose levels, telemedicine for remote patient monitoring, assignment of a peer mentor (having the same age, gender, and ethnicity as the user, as well as being or having been diabetic), and a pedometer and app to use in tracking movement of the user. The platform may identify a number of steps the user should take every day to help lower the user's weight and utilize the pedometer to track the user's daily progress. The pedometer may be configured to provide movement data to the client device. The client device is configured to transmit the movement data to the platform for further analyzing and generation of a new recommendation. The blood testing kit may also be configured to test a user's blood and obtain a blood glucose level that is transmitted to the platform, via the client device, for further analyzing and generation of a new recommendation. The platform is configured to receive new data representing the user's progress after the first recommendation to confirm that the user is obtaining desired results, or alternatively, encourage the user to embark on actions to obtain desired results where results are not favorable based on inaction by the user.

Many of the above-described features and applications are implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

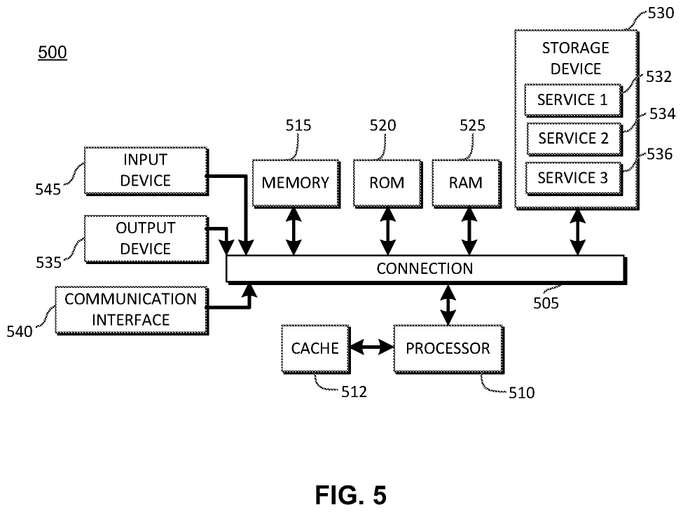

FIG. 5 illustrates an example of a system 500 configured to treat a medical condition using an integrated delivery platform, in accordance with some aspects. A platform which some implementations of the subject technology are implemented may include various types of computer readable media and interfaces for various other types of computer readable media. One or more components of the platform are in communication with each other using connection 505. Connection 505 can be a physical connection via a bus, or a direct connection into processor 510, such as in a chipset architecture. Connection 505 can also be a virtual connection, networked connection, or logical connection.

In some embodiments system 500 is a distributed system in which the functions described in this disclosure can be distributed within a datacenter, multiple datacenters, a peer network, etc. In some embodiments, one or more of the described system components represents many such components each performing some or all of the function for which the component is described. In some embodiments, the components can be physical or virtual devices.

System 500 includes at least one processing unit (CPU or processor) 510 and connection 505 that couples various system components including system memory 515, such as read only memory (ROM) 520 and random access memory (RAM) 525 to processor 510. Computing system 500 can include a cache 512 of high-speed memory connected directly with, in close proximity to, or integrated as part of processor 510.

Connection 505 also couples kiosks to a network through the communication interface 540. In this manner, the kiosks can be a part of a network of computers (such as a local area network (“LAN”), a wide area network (“WAN”), or an Intranet, or a network of networks, such as the Internet.

Processor 510 can include any general purpose processor and a hardware service or software service, such as services 532, 534, and 536 stored in storage device 530, configured to control processor 510 as well as a special-purpose processor where software instructions are incorporated into the actual processor design. Processor 510 may essentially be a completely self-contained computing system, containing multiple cores or processors, a bus, memory controller, cache, etc. A multi-core processor may be symmetric or asymmetric.

To enable user interaction, computing system 500 includes an input device 545, which can represent any number of input mechanisms, such as a microphone for speech, a touch-sensitive screen for gesture or graphical input, keyboard, mouse, motion input, speech, etc. Computing system 500 can also include output device 535, which can be one or more of a number of output mechanisms known to those of skill in the art, and may include, for example, printers and display devices, such as cathode ray tubes (CRT) or liquid crystal displays (LCD). Some implementations include devices such as a touch screen that functions as both input and output devices. In some instances, multimodal systems can enable a user to provide multiple types of input/output to communicate with computing system 500. Computing system 500 can include communications interface 540, which can generally govern and manage the user input and system output. There is no restriction on operating on any particular hardware arrangement and therefore the basic features here may easily be substituted for improved hardware or firmware arrangements as they are developed.

Storage device 530 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read only memory (ROM), and/or some combination of these devices.

The storage device 530 can include software services, servers, services, etc., that when the code that defines such software is executed by the processor 510, it causes the system to perform a function. In some embodiments, a hardware service that performs a particular function can include the software component stored in a computer-readable medium in connection with the necessary hardware components, such as processor 510, connection 505, output device 535, etc., to carry out the function.

It will be appreciated that computing system 500 can have more than one processor 510, or be part of a group or cluster of computing devices networked together to provide greater processing capability.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

It is understood that any specific order or hierarchy of steps in the processes disclosed is an illustration of exemplary approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as a “configuration” does not imply that such configuration is essential to the subject technology or that such configuration applies to all configurations of the subject technology. A disclosure relating to a configuration may apply to all configurations, or one or more configurations. A phrase such as a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims.

Furthermore, to the extent that the term “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A computer-implemented method for generating a recommendation to treat a medical condition, comprising:
analyzing data associated with a user to generate a first recommendation for medically treating a medical condition;
obtaining treatment data from a client device representing a characteristic of the user; and
generating a second recommendation for medically treating the medical condition based on the treatment data.

2. The computer-implemented method of claim 1, further comprising transmitting the first recommendation to client device for storage in the client device.

3. The computer-implemented method of claim 1, further comprising transmitting the second recommendation to client device for storage in the client device.

4. The computer-implemented method of claim 1, further comprising generating a voucher for a prescribed drug to treat the medical condition.

5. The computer-implemented method of claim 4, further comprising monitoring redemption of the voucher.

6. The computer-implemented method of claim 5, further comprising generating a third recommendation modifying a dosage for the prescribed drug.

7. The computer-implemented method of claim 6, further comprising issuing a second voucher based on the third recommendation.

8. The computer-implemented method of claim 1, wherein the first recommendation includes at least one of a drug prescription, testing kit, and peripheral.

9. A non-transitory computer-readable medium comprising instructions, the instructions, when executed by a computing system, cause the computing system to:
analyze data associated with a user to generate a first recommendation for medically treating a medical condition;
obtain treatment data from a client device representing a characteristic of the user; and
generate a second recommendation for medically treating the medical condition based on the treatment data.

10. The non-transitory computer-readable medium of claim 9, wherein the instructions further cause the computing system to transmit the first recommendation to client device for storage in the client device.

11. The non-transitory computer-readable medium of claim 9, wherein the instructions further cause the computing system to transmit the second recommendation to client device for storage in the client device.

12. The non-transitory computer-readable medium of claim 9, wherein the instructions further cause the computing system to generate a voucher for a prescribed drug to treat the medical condition.

13. The non-transitory computer-readable medium of claim 12, wherein the instructions further cause the computing system to monitor redemption of the voucher.

14. The non-transitory computer-readable medium of claim 13, wherein the instructions further cause the computing system to generate a third recommendation modifying a dosage for the prescribed drug.

15. The non-transitory computer-readable medium of claim 14, wherein the instructions further cause the computing system to issue a second voucher based on the third recommendation.

16. A system comprising:
a processor; and
a non-transitory computer-readable medium storing instructions that, when executed by the system, cause the system to:
analyze data associated with a user to generate a first recommendation for medically treating a medical condition;
obtain treatment data from a client device representing a characteristic of the user; and
generate a second recommendation for medically treating the medical condition based on the treatment data.

17. The system of claim 16, wherein the instructions further cause the system to transmit the first recommendation to client device for storage in the client device.

18. The system of claim 16, wherein the instructions further cause the system to generate a voucher for a prescribed drug to treat the medical condition.

19. The system of claim 18, wherein the instructions further cause the system to monitor redemption of the voucher.

20. The system of claim 19, wherein the instructions further cause the system to generate a third recommendation modifying a dosage for the prescribed drug.
